(19) United States
(12) Patent Application Publication
(10) Pub. No.: US 2013/0324859 A1
(43) Pub. Date: Dec. 5, 2013

(54) METHOD FOR PROVIDING INFORMATION FOR DIAGNOSING ARTERIAL STIFFNESS

(71) Applicants: Seung Hun Park, Suwon-si (KR); Dae Geun Jang, Sangju-si (KR); Min Soo Hahn, Daejeon (KR); Umar Farooq, Yongin-si (KR)

(72) Inventors: Seung Hun Park, Suwon-si (KR); Dae Geun Jang, Sangju-si (KR); Min Soo Hahn, Daejeon (KR); Umar Farooq, Yongin-si (KR)

(73) Assignee: University-Industry Cooperation Group of Kyung Hee University, Gyeonggi-do (KR)

(21) Appl. No.: 13/990,357

(22) PCT Filed: Oct. 7, 2011
(86) PCT No.: PCT/KR11/07452

§ 371 Date: Aug. 6, 2013

(30) Foreign Application Priority Data
Nov. 29, 2010 (KR) 10-2010-0119940

(51) Int. Cl. A61B 5/02; A61B 5/00; A61B 5/024

(52) U.S. Cl. CPC A61B 5/02007; A61B 5/02416; A61B 5/7278
USPC 600/479

(57) ABSTRACT

This invention provides a method for assessing arterial stiffness noninvasively using photoplethysmography. The method of the invention for assessing arterial stiffness using photoplethysmography comprises: a user information input step, characteristic point extraction step, and arterial stiffness assessment step. In particular, the characteristic point extraction step includes the correction of the characteristic points, and the arterial stiffness assessment step includes the result of performing multiple linear regression analysis using the baPWV (brachial-ankle pulse wave velocity) value. In addition, according to this invention, arterial stiffness assessment, which was previously an expensive procedure which the user could only obtain at a specialized institution, can be carried out at low cost in the course of daily life, e.g. at home or at work, and can thus be applied in the u-healthcare and home health management service environments.

### BACKGROUND OF THE INVENTION

This invention relates to a method of providing information for diagnosing arterial stiffness at low cost and non-invasively, using photoplethysmography; more specifically, it relates to a method of providing information for diagnosing arterial stiffness wherein after first extracting feature parameters from a photoplethysmography and its second derivative waveform, a linear regression equation for assessing arterial stiffness is extracted by conducting multiple regression analysis, and on this basis, the user's vascular stiffness and vascular aging are assessed and feedback provided.

Cardiovascular conditions have been increasing recently due to the Westernization of eating habits and simple repetitive habits of life. According to a 2009 report by the National Statistical Office of Korea, among all causes of death, the rate of death due to cardiovascular disease was second only to the rate of death due to malignant neoplasms (cancer). Further, according to the statistics of the American Heart Association, approximately 80 million Americans, or about ⅓ of the entire population, are reported to have one or more cardiovascular diseases. Cardiovascular diseases are thus becoming an important social issue both in Korea and worldwide, and the world is growing increasingly aware of this.

Recent research has found that the higher a person's arterial stiffness index is, the higher is that person's probability of suffering from cardiovascular disease. Further, in the case of patients with end-stage renal disease, it has been reported that arterial stiffness can be used as a predictive factor for cardiovascular mortality. Expanding on this, arterial stiffness is a salient prognostic factor for cardiovascular disease, and therefore morbidity of cardiovascular disease can be prevented through ongoing arterial stiffness management.

Various methods have been introduced for measuring arterial stiffness of today's patients. A representative example is the method of using pulse wave velocity. This method is based on the fact that the rate of movement of the pulse wave is accelerated as the blood vessels stiffen and their capacity to store blood is degraded. This is frequently used in clinical settings, due to its enabling measurement of arterial stiffness noninvasively and at a relatively low cost. Other methods that have been introduced involve using ultrasound or MRI to calculate the elastic modulus, Young's modulus, arterial distensibility, and arterial compliance, and calculating arterial stiffness on this basis. However, although these methods yield relatively accurate measurements, they have the disadvantages of high price and the need for a resident specialist to manipulate the apparatus.

Recently, in order to resolve the aforementioned problems, attention has been given to arterial stiffness assessment using photoplethysmography; various feature parameters for this have been proposed. Representative examples of this include the augmentation index, obtained by dividing the difference in amplitude between the pulse wave signal and dicrotic wave by the amplitude of the pulse wave signal; the stiffness index, obtained by dividing the height of the user by the reflected wave arrival time; and the incisura index, obtained by dividing the difference between the pulse signal and incisura amplitudes by the pulse signal amplitude. According to the results of many previous studies, these feature parameters have been reported to have a statistically significant correlation to arterial stiffness.

The second derivative waveform, the signal obtained by taking the second derivative of the photoplethysmogram, has been suggested as another approach for assessing arterial stiffness using photoplethysmography. The second derivative waveform has five broad characteristic points; arterial stiffness can be assessed using their relative size. In particular, the vascular aging indices (b-c-d-e)/a and (b-c-d)/a are known to have a statistically significant correlation to arterial stiffness.

The majority of methods of the prior art for assessing arterial stiffness using photoplethysmography emphasize the correlation between the aforementioned feature parameters and arterial stiffness, and focus excessively on assessing their statistical significance. This indicates that an assessment of arterial stiffness using photoplethysmographic feature parameters can yield statistically significant results. However, in conducting actual assessments of arterial stiffness, there are limits to the extent to which arterial stiffness can be assessed using a single feature parameter; this problem impacts the accuracy, reproducibility and reliability of arterial stiffness measurements made using photoplethysmography.

Therefore, there is an urgent need for a photoplethysmography-based technology for arterial stiffness assessment using one or more feature parameters, whereby measurement can be performed at low cost regardless of time and place, there is no need to have a specialist in residence, and cardiovascular disease can be managed on an ongoing basis.

### SUMMARY OF THE INVENTION

The objective of this invention is to provide a method whereby arterial stiffness can be assessed non-invasively and without restrictions of time and place using photoplethysmography, which enables relatively straightforward measurement.

Another objective of this invention is to provide a method of managing arterial stiffness, whereby ongoing management of cardiovascular disease is possible at relatively low cost, and biofeedback for this can be provided effectively.

Having been devised in order to resolve the above-described problems of the prior art, the method of this invention for providing non-invasive arterial stiffness assessment using the user's photoplethysmogram comprises: a signal processing step wherein parameters for assessing arterial stiffness are extracted from the user's photoplethysmogram; a statistical analysis step wherein a predictive equation whereby arterial stiffness can be assessed is extracted by statistical processing using the parameters extracted in said signal processing step; and a step wherein the user's arterial stiffness is assessed using the regression equation extracted in said statistical analysis step, and the results are provided as effective feedback to the user.

In addition, said signal processing step comprises: a second derivative waveform extraction step for extracting the user's second derivative waveform of photoplethysmogram (SDPTG); a valid pulse wave signal extraction step wherein only the valid pulse wave signal is extracted from said user's photoplethysmogram, excluding noise components; a pulse wave segmentation step wherein said user's photoplethysmogram is segmented periodically; a pulse waveform classification step, wherein pulse waveforms are classified based on said photoplethysmogram and second derivative waveform; and a feature parameter extraction step wherein characteristic points and arterial stiffness assessment parameters are extracted from said photoplethysmogram and second derivative waveform.

In addition, said statistical analysis step comprises: a regression equation extraction step wherein multiple linear regression analysis is conducted using said user information and extracted feature parameters, and the arterial stiffness assessment equation is extracted as a result thereof.

In addition, said second derivative waveform extraction step comprises: a step wherein, in order to remove the ultra-high frequency wave component arising within said photoplethysmogram due to quantization, at least one of a linear fitting algorithm, a moving average filter, and a low pass filter are applied; and a step wherein the second derivative waveform is extracted by using a differential operator and lowpass filter to at least one of said photoplethysmogram, the first derivative of photoplethysmogram, and the second derivative waveform.

In addition, said valid pulse wave signal extraction step comprises: a preprocessing step to verify the validity of the pulse wave signal, wherein the size of the analysis window is calculated using at least one of: an average magnitude difference function (AMDF) and an autocorrelation function; a step wherein in order to resolve the problems of pitch doubling and pitch halving of the AMDF and autocorrelation function, by using at least one of a moving average filter and a median filter are used; and a step wherein the invalid signal range is detected by using at least one of the minimum value of the signal included in said analysis window and the amount of change therein, the amplitude of the signal (difference between maximum and minimum), the number of peaks, and the level crossing rate.

In addition, said pulse wave segmentation step comprises: a step wherein the pulse wave signal is segmented by using at least one of pulse length, pulse height, pulse area, and change in pulse onset, obtained from said photoplethysmogram; and a step wherein in order to calculate the threshold value of said feature parameters, a signal-adaptive threshold value is determined based on prior knowledge of each feature parameter.

In addition, said pulse waveform classification step comprises: a step wherein pulse waveforms are classified quantitatively using at least one or more of whether a dicrotic wave occurred in said photoplethysmogram, and the location of the dicrotic wave; and a step wherein the pulse waveform of the second derivative waveform is classified based on said second derivative waveform, using at least one or more of whether a “b” wave occurred and the amplitude thereof, whether a “c” wave occurred and the coding thereof, and whether a “d” wave occurred and the amplitude thereof

In addition, said characteristic point extraction step comprises: a step wherein at least one or more of the pulse onset, pulse peak, incisura, and dicrotic wave of the photoplethysmogram are extracted, discriminatively applying a characteristic point extraction method according to the waveform determined in said waveform classification step; and a step wherein at least one or more of the initial positive wave, early negative wave, late upsloping wave, late downsloping wave, and diastolic positive wave of the second derivative waveform are extracted, differentially applying a characteristic point extraction method according to the waveform determined in said waveform classification step.

In addition, said feature parameter extraction step comprises: a step wherein the augmentation index, reflected wave arrival time, peak-to-onset time interval, peak-to-incisura time interval, and vascular aging index are calculated using at least one or more of the onset, peak, incisura and dicrotic wave of the photoplethysmogram, obtained in said characteristic point extraction step, and at least one or more of the initial positive wave, early negative wave, late upsloping wave, late downsloping wave, and diastolic positive wave of the second derivative waveform, and at least one or more thereof is used as a predictive parameter for arterial stiffness; and a step wherein the values of said feature parameters are corrected using at least one or more of normalization using the pulse wave length, Bazett's formula, Fridericia's formula, Hodge formula and a linear regression equation as a predictive parameter for arterial stifness.

In addition, said regression equation extraction step comprises: a step wherein a linear regression equation such as the following is extracted by multiple linear regression analysis of the baPWV value that quantitatively represents arterial stiffness, and at least one or more parameters (A, B, C) from among said feature parameters and user information (age, sex, height, weight, and BMI).

Y=α×A+β or

Y=α×A+β×B+γ or

Y=α×A+β×B+γ×C+δ

In addition, said feedback step comprises: a step wherein the result of arterial stiffness assessment extracted using said linear regression equation is compared with the reference value for the respective sex and age, and biofeedback is provided to said user by calculating vascular age on the basis thereof.

According to this invention, the user can monitor his or her own arterial stiffness status on an ongoing basis; the user's awareness of cardiovascular disease is heightened by providing feedback based on a comparison with the standard value for the respective sex and weight; and the user can reduce the morbidity of cardiovascular disease through ongoing prevention and management.

In addition, according to this invention, a new type of cardiovascular disease management service can be provided that can be used widely in the u-healthcare and home health management service environments, as it would enable low-cost assessment of arterial stiffness without restrictions of place and time.

### BRIEF DESCRIPTION OF THE DRAWINGS

FIG. 1 is a flowchart showing one embodiment of the method of this invention for providing information for diagnosis of arterial stiffness, in conceptual form.

FIG. 2 is a flow chart showing in detail one embodiment of the pulse characteristic point extraction step (S120) depicted in FIG. 1.

FIG. 3a is a flow chart showing one embodiment of the linear fitting algorithm of the second derivative waveform extraction step (S210) depicted in FIG. 2.

FIG. 3b is a flow chart showing one embodiment wherein a second derivative waveform has been extracted using Formula 1 and the linear fitting algorithm depicted in FIG. 3a.

FIG. 4 shows the valid signal extraction criteria used in the valid signal range extraction step (S220) depicted in FIG. 2.

FIG. 5 shows the segmentation criteria used in the photoplethysmogram segmentation step (S230) depicted in FIG. 2.

FIG. 6a shows the characteristic points and feature parameters of the photoplethysmogram.

FIG. 6b shows the characteristic points and feature parameters of the second derivative waveform.

FIG. 7a shows the four waveforms of the photoplethysmogram.

FIG. 7b shows the seven waveforms of the second derivative waveform.

FIG. 8a shows one embodiment of the results of extraction of the characteristic points and feature parameters of the photoplethysmogram and second derivative waveform, according to this invention.

FIG. 8b shows one embodiment of the result of arterial stiffness assessment according to this invention.

### DESCRIPTION OF SPECIFIC EMBODIMENTS

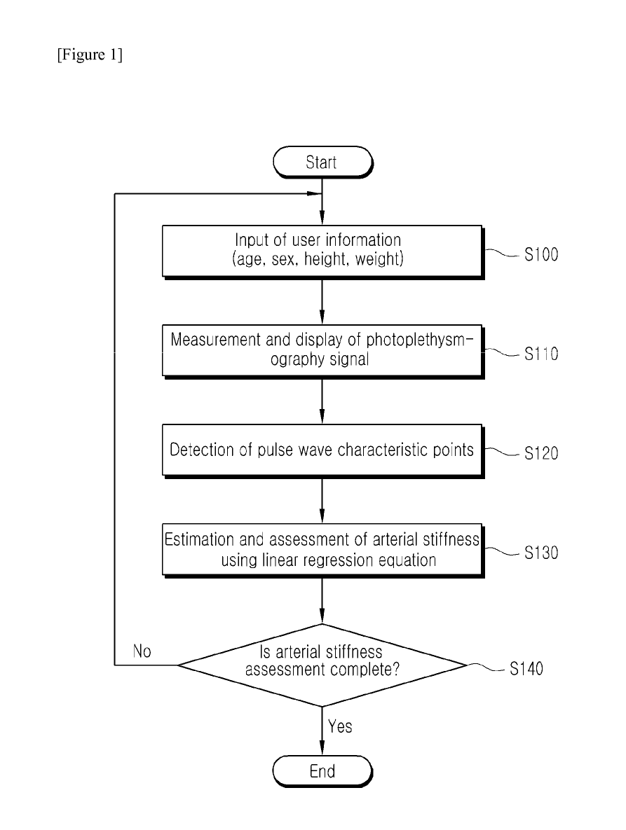

Preferred embodiments of the method according to this invention for providing information for diagnosis of arterial stiffness will now be explained with reference to FIGS. 1 through 8b. In the process, the thickness of lines or size of components in the drawings may be exaggerated for clarity and convenience of explanation. In addition, the terms described below are defined with reference to the functionality of this invention; this may differ depending on the intentions or habits of the user or operator. Therefore, the definitions of these terms must be described on the basis of the overall content of this specification.

FIG. 1 is a flow chart showing, in conceptual form, one embodiment of the method according to one aspect of this invention for providing information for diagnosis of arterial stiffness.

First, prior to measuring the photoplethysmogram, the user information for age, sex, height and weight is entered (S100). Generally, the degree of arterial stiffening differs depending on age and sex, and body condition including height and weight is also known to have an impact. Therefore, in a method of arterial stiffness assessment using photoplethysmography, biometric information is salient as an independent predictive factor, and a user interface must be provided for entering it.

When user information has been entered, the photoplethysmogram is obtained at the user's fingertip (S110). In order to properly assess arterial stiffness, accurate measurement of the photoplethysmogram is needed. Therefore, it is important that the user hold a stable position while the photoplethysmogram is obtained, and that exposure to outside noise (light sources, movement noise, etc.) be avoided.

The user's photoplethysmogram can be obtained by diverse methods. A light-emitting optical sensor and a light-receiving photoreceptor are needed in order to measure the photoplethysmogram. When the optical signal emitted by the optical sensor strikes the fingertip, a portion of either penetrates or reflects and is received as input by the photoreceptor, and the photoreceptor converts the input light to an electrical signal to measure the photoplethysmogram. Generally, the optical sensor used for measuring the photoplethysmogram is either a red LED optical sensor having a wavelength of 660 nm or an infrared LED sensor having a wavelength of 805 nm.

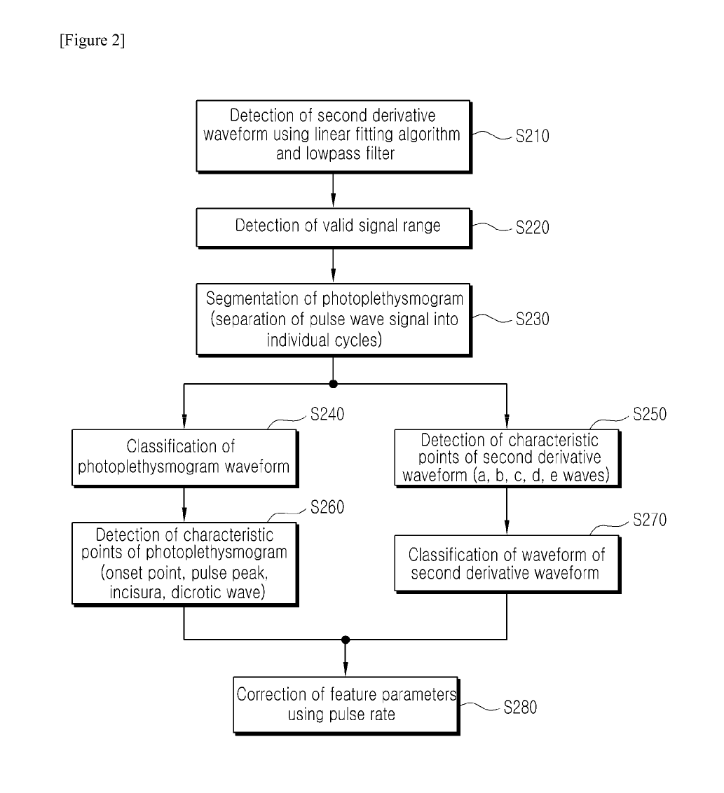

FIG. 2 is a flow chart showing in detail one embodiment of the pulse characteristic point extraction step (S120) depicted in FIG. 1.

The various characteristic points and feature parameters for assessment of arterial stiffness are extracted after measuring the user's photoplethysmogram (S120).

FIG. 2 shows an one embodiment of the extraction of the characteristic points and feature parameters in detail; it comprises: a second derivative waveform detection step (S210) wherein the second derivative waveform is calculated using a lowpass filter and linear fitting algorithm; a valid signal detection step (S220) for removing noise and invalid signal ranges from the original signal; a pulse wave segmentation step (S230) wherein the pulse wave signal for one cycle is segmented for characteristic point extraction; a waveform classification step (S240, S270) wherein the waveforms of the photoplethysmogram and second derivative waveform are classified; a characteristic point extraction step (S250, S260) wherein the characteristic points of the photoplethysmogram and second derivative waveform are extracted; and a feature parameter correction step (S280) for correcting the feature parameters that are influenced by pulse rate.

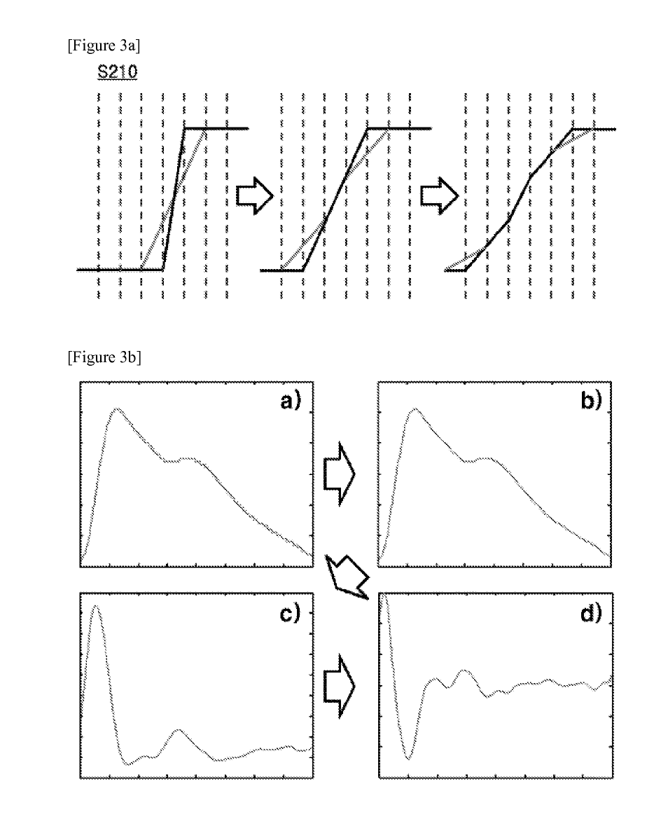

FIG. 3a is a flow chart showing one embodiment of the linear fitting algorithm of the second derivative waveform extraction step depicted in FIG. 2; FIG. 3b is a flow chart showing one embodiment wherein a second derivative waveform has been extracted using Formula 1 and the linear fitting algorithm depicted in FIG. 3a (S210). In FIG. 3b, a) is the original signal, b) is the result of linear fitting, c) is the first derivative, and d) is the second derivative waveform.

First, in preprocessing, various signals are calculated in order to extract the exact characteristic points; a linear fitting algorithm is applied for this purpose. For the linear fitting algorithm, linear smoothing of the high-frequency component is applied as shown in FIG. 3a. The initial left-side graph shows the photoplethysmogram signal collected from the measurement apparatus; proceeding to the left, embodiments are depicted that have passed through the linear fitting algorithm.

The sequence in which the linear fitting algorithm is performed is as follows. First, the slope is calculated using the difference between adjacent samples. Based on the calculated slope information, the components are calculated as zero-slope or non-zero-slope. Each sample of the input signal is classified broadly into four states, depending on the slope: (slope=0, slope=0), (slope=0, slope≠0), (slope≠0, slope=0) and (slope≠0, slope≠0). If there is a zero-slope component in the sample, the sample value for the relevant range is altered using a first-order linear equation. Here the first-order linear equation is calculated using the zero-slope component and the values of two adjacent samples.

This linear fitting algorithm can forestall the nonlinear time delay that could otherwise arise during lowpass filtering, by removing the high-frequency component.

The second derivative waveform is extracted using the third graph, on the right, which has passed through the linear fitting algorithm.

The linear fitting algorithm of FIG. 3 and the lowpass filter of Formula 1 are used to extract the user's second derivative waveform.

$$y [ n ] = {\sum }_{k = 0}^{N} h [ k ] x [ n - k ] \qquad [ Formula 1 ]$$

In Formula 1, y[n] and x[n] respectively represent the result signal that has passed through the low pass filter, and the input signal. h[k] and N respectively represent the filter coefficient and the order of the low pass filter.

The photoplethysmogram obtained by the apparatus may include signals distorted e.g. by user movement, introduction of external light sources, and slight movements of the sensor. These noise and distortion signals reduce the accuracy and reliability of the arterial stiffness measurement; therefore, it is necessary to extract only the valid pulse signal from the original signal that contains noise and distortion.

To extract the valid pulse signal range, first, the size of the analysis window needs to be calculated. To this end, the pulse signal for one cycle is roughly estimated using the normalized autocorrelation function of Formula 2.

$${R}_{n} ( τ ) = \frac{\sqrt{{\sum }_{n = 0}^{N - 1} s ( n ) s ( n + τ )}}{\sqrt{{\sum }_{n = 0}^{N - 1} {s}^{2} ( n + τ )}} \qquad [ Formula 2 ]$$

In Formula 2, s(n) and Rₙ(t) respectively represent the pulse wave signal and the autocorrelation signal thereof The approximate pulse wave cycle can be extracted by extracting from the autocorrelation signal the first peak value that exceeds a specific threshold value. Here, at least one of a moving average filter and a median filter are used to resolve the problems of pitch doubling or pitch halving that arise when using the autocorrelation function,

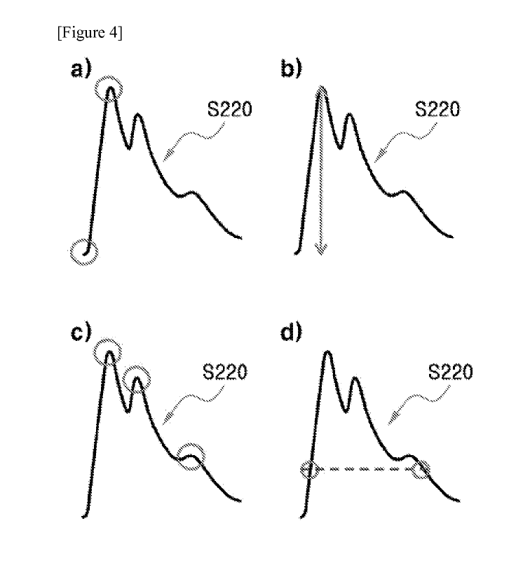

FIG. 4 shows the valid signal extraction criteria used in the valid signal range extraction step (S220) depicted in FIG. 2. Here, a) shows the maximum and minimum values and the changes between them; b) shows the difference between the maximum and minimum values and the changes therein; c) shows the number of peaks; and d) shows the level crossing rate.

FIG. 4 as shown relates to main processing; the size of the analysis window for determining the valid signal range in the measured pulse wave signal is calculated using an autocorrelation function or AMDF function. When thus using the autocorrelation function or AMDF function, the number of operations required will depend on the size of the analysis window. Therefore, a multi-level center clipper is used in order to resolve the overflow and computational processing speed issues arising with increased computational load, and a median filter is used to correct the problem of pitch doubling and pitch halving. It is then determined whether the range in question is a valid signal range or an invalid signal range, using the amplitude of the signal within the calculated analysis window, the number of peaks, the level crossing rate, the highest value and the lowest value, and the degree of change in these.

After calculating the size of the analysis window, the information depicted in FIG. 4 is used to verify the validity of the signal within the analysis window. Because the size of the analysis window encompasses one cycle of the pulse wave signal, the validity of the signal can be verified using the threshold values for the range that one cycle of the pulse wave signal can have. Because the absolute value of the photoplethysmogram will vary depending on the measurement apparatus and the method of signal processing, it is important that the threshold values be determined using relative indices such as the relative amplitude of the pulse wave or the relative time interval of the pulse wave cycle.

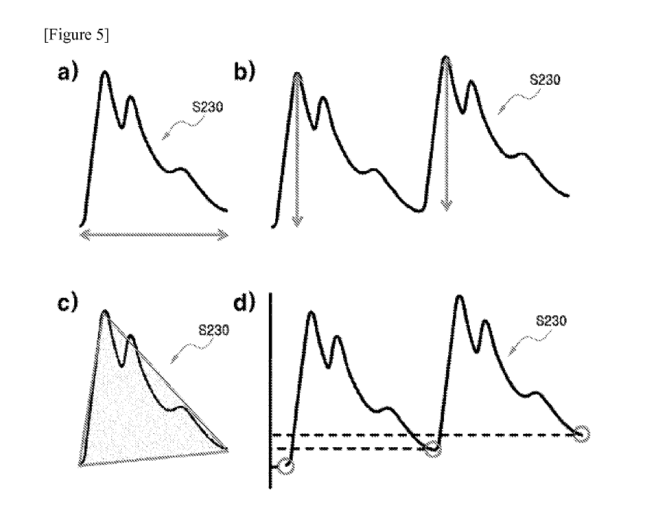

FIG. 5 shows the segmentation criteria used in the photoplethysmogram segmentation step (S230) depicted in FIG. 2. Here, a) shows the pulse wave length, b) shows the degree of change in pulse wave amplitude, c) shows the pulse wave area, and d) shows the degree of change in onset.

Referring to FIG. 5, pulse wave segmentation is performed after the valid signal range has been extracted via the above-described process. Here, pulse wave segmentation involves the division of a pulse wave signal, comprising several cycles, into individual cycles. To this end, a function such as the above-mentioned autocorrelation function or AMDF is used to determine the initial threshold values. When the initial threshold values have been determined, the following parameters are used to extract the exact onset point, and the extracted onset point is used to segment the pulse wave signal.

The information depicted in FIG. 5 is used to segment the photoplethysmographic signal present within the valid signal range into individual cycles. The segmentation of the photoplethysmogram is the same as the process of detecting the onset point; therefore, the photoplethysmogram segmentation process can be regarded as the onset point detection process. To accomplish this, all points where a notch appears are compared to the threshold values of the criteria depicted in FIG. 5, and a notch that satisfies all criteria is regarded as an onset point of the photoplethysmographic signal. The threshold value for each of the criteria is then characterized by adapting to the correct value for the signal, based on prior knowledge such as statistical indices. In addition, the newly-calculated threshold values are used as prior knowledge for the pulse wave segmentation of the next cycle; the suitable threshold values for the signal are determined automatically. Characteristic points of the pulse wave signal for each cycle can be extracted using all the onset points extracted by the above method.

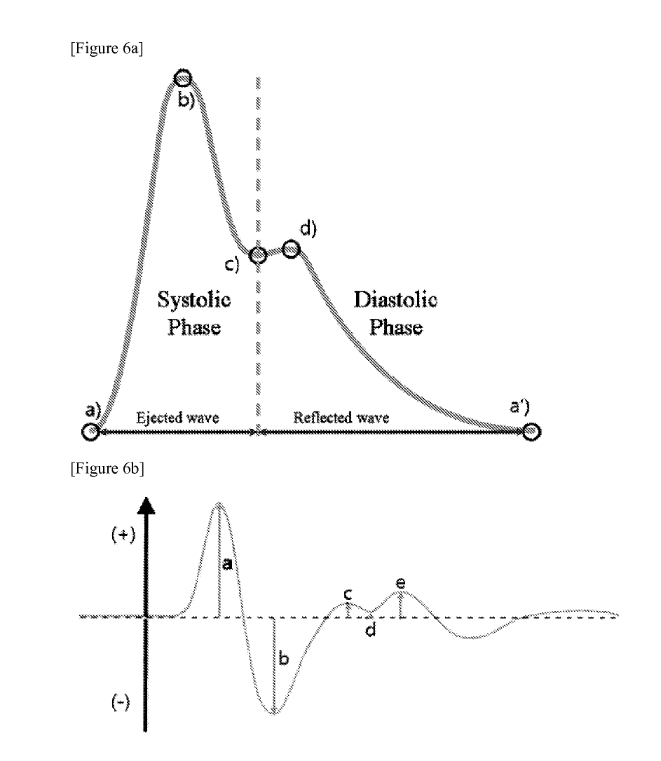

FIG. 6a shows the characteristic points and feature parameters of the photoplethysmogram. Here a) and a)′ show the onset points, b) shows the peak, c) shows the incisura, and d) shows the dicrotic wave.

FIG. 6b shows the characteristic points and feature parameters of the second derivative waveform. Here a) shows the initial positive wave, b) shows the early negative wave, c) shows the late upsloping wave, d) shows the late downsloping wave, and e) shows the diastolic positive wave.

First, as the left ventricle contracts, the internal pressure of the left ventricle increases and the aortic valve is opened. As the aortic valve is opened, the blood from the left ventricle is ejected via the aortic arch, and this corresponds to the onset point (a in FIG. 6a). Thereafter the blood is rapidly drawn in from the left ventricle to the aortic arch, and the intravascular pressure and vascular capacity reach a maximum (b in FIG. 6a). This is because, thereafter, the pressure and capacity are influenced by the reduction in blood volume. Thereafter, the right ventricle contracts and the left ventricle expands, as the aortic value is closed. The point at which the aortic valve closes is the incisura (c in FIG. 6a). After the aortic valve has closed, the intraarterial pressure and volume increase slightly; this corresponds to the dicrotic wave (d in FIG. 6a). From the dicrotic wave to the onset of the next cycle (a′ in FIG. 6a), the left ventricle expands, receiving blood from the left atrium.

With regard to the second derivative waveform, there are 5 characteristic points; typically a, c and e waves form convex curves in the positive direction, while b and d waves form convex curves in the negative direction. The a waves and b waves are the components that first respond in the blood vessels to the ejection of blood from the left ventricle, and therefore the b/a ratio represents vascular distensibility. In addition, the d/a ratio represents the strength of the wave reflected from the extremities, and a reduction in the d/a ratio represents an increase in the reflected wave. The (b-c-d-e)/a index is conventionally used to assess vascular elasticity and stiffness.

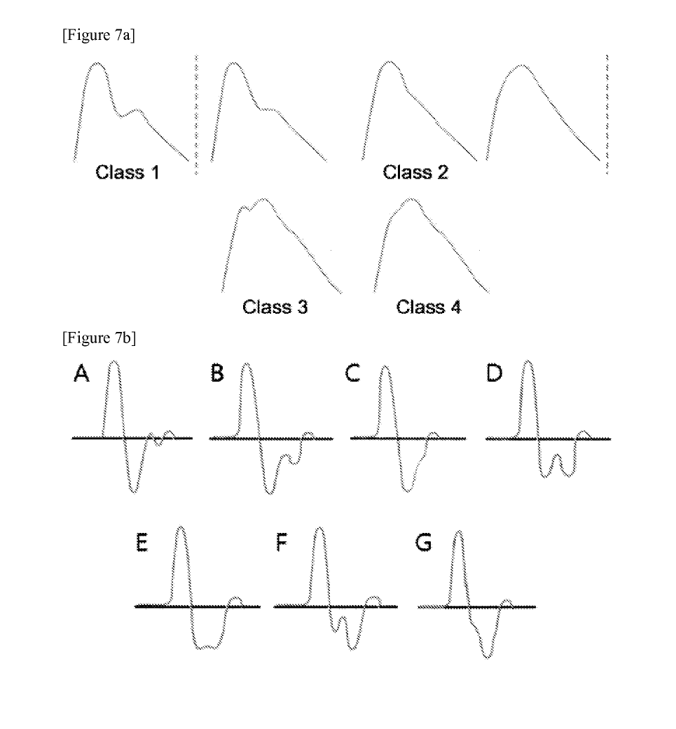

FIG. 7a shows the four types of waveforms of the photoplethysmogram (Class 1-Class 4), and FIG. 7b shows the seven types of waveforms of the second derivative waveform (Class A-Class G).

Referring to FIGS. 7a and 7b, one thing that is necessary in order to extract accurate characteristic points is to accurately classify the waveforms. Because the method of extraction differs depending on the waveform, accurate waveform classification is critical. To this end, it is preferable that PTG signals be broadly classified into three types depending on the position of the dicrotic wave and the incisura, and that SDPTG signals be broadly classified into seven types depending on the codes of the characteristic points.

According to the findings of previous research, with increasing age and coronary artery disease, the incidence of Class 2 in FIG. 7a increases, and it has been reported that among male myocardial infarction patients 65-74 years old, patients exhibiting the Class 2 waveform of FIG. 7a are four times more numerous than patients exhibiting the Class 1 waveform of FIG. 7a (Dawber, Thomas, McNamara, 1973). It has also been reported that the more prevalent Class 4 of FIG. 6a is over Class 1 of FIG. 6a, the more attenuated the incisura becomes (Millasseau, Ritter, Takazawa, Chowienczyk, 2006). With regard to the second derivative waveforms shown in FIG. 7b, it has been reported that the incidence of Classes E, F and G increases with age.

To extract the characteristic points of the photoplethysmogram depicted in FIG. 6a, the pulse waveform classification criteria of FIG. 7a are used. After classifying the pulse waveform using the occurrence or nonoccurrence, and position, of the dicrotic wave, a characteristic point extraction algorithm is applied based on the pulse waveform. First, the peak location having the highest value within the pulse wave signal of a single cycle is extracted as the pulse peak (b in FIG. 6a). If the waveform is Class 1 or Class 3 of FIG. 7a, the peaks corresponding to the onset point and pulse peak, or pulse peak and onset point, are used to extract the dicrotic wave (d in FIG. 6a) and incisura (c in FIG. 6a). In contrast, if the waveform is Class 2 or Class 4 of FIG. 7a, then after extracting the inflection point using the second derivative waveform, this is used to extract the dicrotic wave and incisura.

The feature parameters for assessing arterial stiffness using said extracted characteristic points of the photoplethysmogram are defined as follows (FIG. 6a):

TABLE 1

| Feature parameter | Definition | Feature parameter | Definition |
|---|---|---|---|
| Augmentation | (b − a)/a | Stiffness Index | Height/reflected |
| Index (AI) | | (SI) | wave arrival time |
| Incisura Index | (b − c)/a | Reflected Wave | b − d time interval |
| (CI) | | Arrival Time (RT) | |
| Upstroke Time | a − b time | Ejection Time | a − c time interval |
| (UT) | interval | (ET) | |
| Peak-to-Onset | b − a′ time | Peak-to-Incisura | b − c time interval |
| (P2O) time | interval | (P2I) time interval | |
| interval | | | |

To extract the characteristic points of the second derivative waveform shown in FIG. 6b, first, the peak point having the greatest value is extracted from the second derivative waveform for one cycle as the initial positive wave (b in FIG. 6b). After extracting the initial positive wave, the diastolic positive wave (e in FIG. 6b) is extracted using the peak envelope. The extracted initial positive wave and diastolic positive wave are used to determine the range wherein the initial negative wave, late upsloping wave, and late downsloping wave may appear. The initial negative wave is determined by extracting the smallest value from among the signals contained within said range, and the late upsloping wave and late downsloping wave are extracted using the peak and notch occurring between the initial positive wave and initial negative wave, and between the initial negative wave and diastolic positive wave. The waveform is classified using the characteristic points of the extracted second derivative waveform and the waveform classification criteria of FIG. 7b.

The feature parameters for assessing arterial stiffness using said extracted characteristic points of the second derivative waveform are defined as follows (FIG. 6b):

TABLE 2

| Feature | | | |
|---|---|---|---|
| parameter | Definition | Feature parameter | Definition |
| Vascular | (b − c − d − e)/a | Vascular aging index 2 | (b − c − d)/a |
| aging index 1 | | | |
| Vascular | (b − c)/a | Initial negative wave/ | b/a |
| aging index 3 | | initial positive wave | |
| Late | c/a | Late upsloping wave/ | d/a |
| downsloping | | initial positive wave | |
| wave/initial | | | |
| upsloping wave | | | |

Arterial stiffness can be assessed using the feature parameters defined in Tables 1 and 2 above. However, because the reflected wave arrival time, upstroke time, ejection time, peak-to-onset time interval, and peak-to-incisura time interval are all influenced by the pulse rate, post-processing is needed to correct for this.

[Formula 3]

QT_{c}=QT(HR/60)^{1/2}=QT(HR)^{-1/2} Bazett's formula:

[Formula 4]

QT_{c}=QT(HR)^{1/3}=QT(RR)^{-1/3} Fridericia's formula:

The effect of the pulse rate on said feature parameters is corrected using Formula 3, Formula 4, and a linear regression equation. The method of using the linear regression equation specifically involves analyzing the correlation between the pulse rate and the feature parameters to calculate the linear regression equation, and then using this to correct for the effect of the pulse rate; this has relatively good performance.

The arterial stiffness estimation and assessment step (S130) using the linear regression equation, depicted in FIG. 1, involves the assessment of arterial stiffness using the extracted feature parameters and user information. First, multiple linear regression analysis is conducted using the feature parameters, user information, and arterial stiffness measurement results. The linear regression equation for predicting arterial stiffness is calculated using the user information and feature parameters that have the greatest correlation to the arterial stiffness measurement results.

Y=α×A+β or

Y=α×A+β×B+γ or

Y=α×A+β×B+γ×C+δ [Formula 5]

Formula 5 shows the general form of the linear regression equation for assessing arterial stiffness, where Y represents the arterial stiffness measurement result, and A, B, C represent the feature parameters and user information used to assess arterial stiffness. In Formula 5, Y represents the arterial stiffness measurement result, and A, B, C represent the feature parameters and user information used to assess arterial stiffness. In addition, α, β, γ, δ represent the coefficients of the linear regression equation. The coefficients of the linear regression equation of Formula 5 for assessing arterial stiffness will vary depending on sex and age, and the feature parameters and user information that are used will also differ.

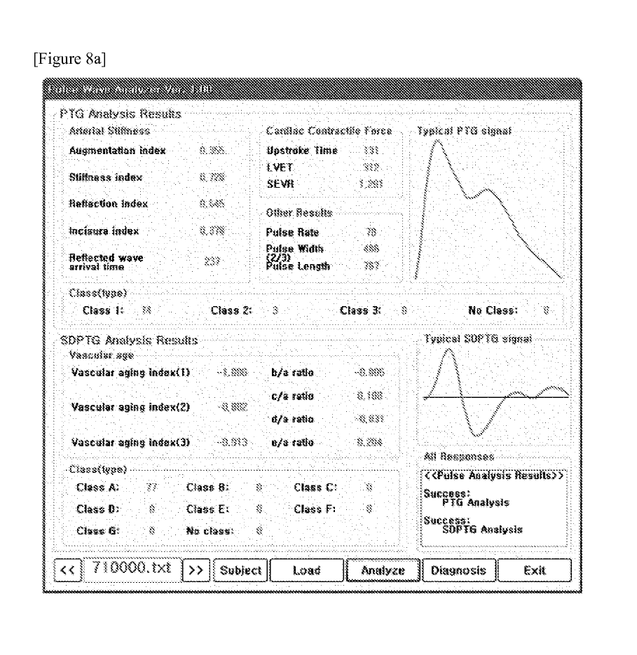

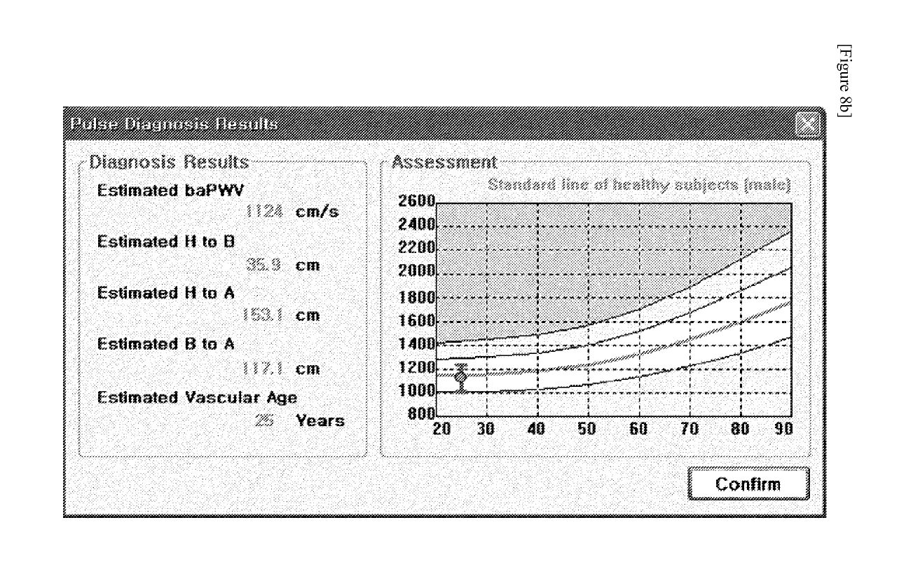

FIG. 8a shows one embodiment of the results of extraction of the characteristic points and feature parameters of the photoplethysmogram, according to this invention; FIG. 8b shows one embodiment of the result of arterial stiffness assessment using the photoplethysmogram.

First, user sex, age, height, and weight are entered, and the photoplethysmogram is obtained at the user's fingertip. The characteristic points and feature parameters are calculated from the obtained photoplethysmogram and second derivative waveform, and the results thereof are shown to the user (FIG. 8a). The number of waveforms (S240, S270) classified in the waveform characteristic point extraction step (S120) depicted in FIG. 1 is output and the waveform most frequently extracted is shown as the user's representative waveform. Using the input user information and extracted feature parameters, arterial stiffness is assessed, and upon comparing this to the reference value for the given age and sex, feedback is given to the user (FIG. 8b).

The method of this invention for assessing arterial stiffness based on photoplethysmography, as described above, enables relatively straightforward use and measurement, unlike the methods of the prior art that requite expert knowledge on the part of the evaluator; because it is not restricted by place or time, it can be applied in the u-healthcare and home health management industries, and it can also be used to improve the health of the elderly and patients requiring ongoing management of cardiovascular disease.

This invention has been described hereinabove with reference to a preferred embodiment, but it will be evident to a person having ordinary skill in the art that this invention can be amended and altered in diverse ways without departing from the idea and scope of this invention as set forth in the claims below.

What is claimed is:

1. A method for providing information for diagnosing arterial stiffness, comprising:
a signal processing step wherein parameters for assessing arterial stiffness are extracted from the user's photoplethysmogram;
a statistical analysis step wherein a predictive equation, whereby arterial stiffness can be assessed, is extracted by statistical processing using the parameters extracted in said signal processing step; and
a step wherein the user's arterial stiffness is assessed using the regression equation extracted in said statistical analysis step, and the results are provided as effective feedback to the user.

2. The method of claim 1 for providing information for diagnosing arterial stiffness, wherein said signal processing step comprises:
a second derivative waveform extraction step for extracting the user's second derivative of photoplethysmogram (SDPTG);
a valid pulse wave signal extraction step wherein only the valid pulse wave signal is extracted from said user's photoplethysmogram, excluding noise components;
a pulse wave segmentation step, wherein said user's photoplethysmogram is segmented into individual cycles;
a pulse waveform classification step, wherein pulse waveforms are classified based on said photoplethysmogram and second derivative waveform; and
a feature parameter extraction step wherein characteristic points and arterial stiffness assessment parameters are extracted from said photoplethysmogram and second derivative waveform.

3. The method of claim 1 for providing information for diagnosing arterial stiffness, wherein said statistical analysis step comprises:
a regression equation extraction step wherein multiple linear regression analysis is conducted using said user information and extracted feature parameters, and the arterial stiffness assessment equation is extracted as a result thereof.

4. The method of claim 2 for providing information for diagnosing arterial stiffness, wherein said second derivative waveform extraction step comprises:
a step wherein, in order to remove the ultra-high frequency wave component arising within said photoplethysmogram due to quantization, at least one of a linear fitting algorithm, a moving average filter, and a low pass filter are applied; and
a step wherein the second derivative waveform is extracted by using a differential operator and lowpass filter to at least one of said photoplethysmogram, the first derivative of photoplethysmography, and the second derivative waveform.

5. The method of claim 2 for providing information for diagnosing arterial stiffness, wherein said valid pulse wave signal extraction step comprises:
a preprocessing step to verify the validity of the pulse wave signal, wherein the size of the analysis window is calculated using at least one of an average magnitude difference function (AMDF) and an autocorrelation function;
a step wherein in order to resolve the problems of pitch doubling and pitch halving of the AMDF and autocorrelation function, by using at least one of a moving average filter and a median filter are used; and
a step wherein the invalid signal range is detected by using at least one of the minimum value of the signal included in said analysis window and the amount of change therein, the amplitude of the signal (difference between maximum and minimum), the number of peaks, and the level crossing rate.

6. The method of claim 2 for providing information for diagnosing arterial stiffness, wherein said pulse wave segmentation step comprises:
a step wherein the pulse wave signal by using at least one of pulse length, pulse height, pulse area, and pulse wave onset point in said photoplethysmogram; and
a step wherein in order to calculate the threshold value of said feature parameters, a signal-adaptive threshold value is determined based on prior knowledge of each feature parameter.

7. The method of claim 2 for providing information for diagnosing arterial stiffness, wherein said pulse waveform classification step comprises:
a step wherein pulse waveforms are classified quantitatively using at least one or more of whether a dicrotic wave occurred in said photoplethysmogram, and the location of the dicrotic wave; and
a step wherein the pulse waveform of the second derivative is classified based on said second derivative, using at least one or more of whether a “b” wave occurred and the amplitude thereof, whether a “c” wave occurred and the coding thereof, and whether a ‘d” wave occurred and the amplitude thereof.

8. The method of claim 2 for providing information for diagnosing arterial stiffness, wherein said characteristic point extraction step comprises:
a step wherein at least one or more of the pulse onset, pulse peak, incisura, and dicrotic wave of the photoplethysmogram are extracted, discriminatively applying a characteristic point extraction method according to the waveform determined in said waveform classification step; and
a step wherein at least one or more of the initial positive wave, early negative wave, late upsloping wave, late downsloping wave, and diastolic positive wave of the second derivative are extracted, differentially applying a characteristic point extraction method according to the waveform determined in said waveform classification step.

9. The method of claim 2 for providing information for diagnosing arterial stiffness, wherein said feature parameter extraction step comprises:
a step wherein the augmentation index, reflected wave arrival time, peak-to-onset time interval, peak-to-incisura time interval, and vascular aging index are calculated using at least one or more of the onset, peak, incisura and dicrotic wave of the photoplethysmogram that were extracted in said characteristic point extraction step, and at least one or more of the initial positive wave, early negative wave, late upsloping wave, late downsloping wave, and diastolic positive wave of the second derivative; and at least one or more thereof is used as a predictive parameter for arterial stiffness; and
a step wherein the values of said feature parameters are corrected using at least one or more of normalization using the pulse wave length, Bazett's formula, Fridericia's formula, Hodge's formula and a linear regression equation as a predictive parameter for arterial stiffness.

10. The method of claim 3 for providing information for diagnosing arterial stiffness, wherein said regression equation extraction step comprises:
a step wherein a linear regression equation such as the following is extracted by multiple linear regression analysis of the baPWV value that quantitatively represents arterial stiffness, and at least one or more parameters (A, B, C) from among said feature parameters and user information (age, sex, height, weight, and BMI):
Y=α×A+β or
Y=α×A+β×B+γ or
Y=α×A+β×B+γ×C+δ
wherein Y represents the result of arterial stiffness assessment, A, B, C represent arterial stiffness assessment parameters, and α, β, γ, δ represent coefficients of the linear regression equation.

11. The method of claim 1 for providing information for diagnosing arterial stiffness, wherein said feedback step comprises:
a step wherein the result of arterial stiffness assessment extracted using said linear regression equation is compared with the reference value for the respective sex and age, and a biofeedback result is provided to said user by calculating the vascular age on the basis thereof.
